(19) United States
(12) Patent Application Publication
(10) Pub. No.: US 2014/0303525 A1
(43) Pub. Date: Oct. 9, 2014

(54) METHOD AND DEVICE FOR NON-INVASIVE ACOUSTIC STIMULATION OF STEM CELLS AND PROGENITOR CELLS IN A PATIENT

(71) Applicant: Balaji Sitharaman, Coram, NY (US)

(72) Inventor: Balaji Sitharaman, Coram, NY (US)

(73) Assignee: The Research Foundation for The State University of New York

(21) Appl. No.: 14/125,244

(22) PCT Filed: Jun. 8, 2012
(86) PCT No.: PCT/US12/41689

§ 371 Date: Jun. 5, 2014

Related U.S. Application Data
(60) Provisional application No. 61/495,741, filed on Jun. 10, 2011.

(51) Int. Cl. A61K 41/00; A61N 5/06; A61N 1/40; A61N 7/00

(52) U.S. Cl. CPC A61K 41/0047; A61N 7/00; A61N 5/062; A61N 1/40; A61N 2007/0013; A61N 2005/0659; A61N 2005/067
USPC 601/2

(57) ABSTRACT

The present invention provides a method for non-invasive acoustic stimulation of stem cells and/or progenitor cells in a patient. The invention also provides a device for non-invasive stimulation of stem cells and/or progenitor cells in the patient by generating and delivering acoustic waves of a suitable frequency and intensity to the stem cells and/or progenitor cells. The method and device of the present invention is useful in enhancing regeneration of bones and other tissues, such as cartilages, muscles, and nerve tissues, in a patient, for treatment of conditions such as bone loss or fracture.

### REFERENCE TO RELATED APPLICATIONS

This application claims priority to U.S. Provisional Application No. 61/495,741, filed Jun. 10, 2011, the content of which is incorporated herein by reference in its entirety.

### FEDERAL FUNDING

This invention was made with government support under grant/contract number OD007394 awarded by the National Institute of Health. The government has certain rights to the invention.

### FIELD OF THE INVENTION

The present invention relates to a method for non-invasive acoustic stimulation of stem cells and/or progenitor cells in a patient. The invention also relates to a device for non-invasive stimulation of stem cells and/or progenitor cells in the patient by generating and delivering acoustic waves of a suitable frequency and intensity to the stem cells and/or progenitor cells.

### BACKGROUND OF THE INVENTION

Marrow, or mesenchymal stromal cells (MSCs), are multipotent cells found predominantly within the bone marrow. MSCs are undifferentiated progenitor cells for various mesenchymal tissues and can differentiate into tissue types including but not limited to bone, cartilage, and adipose. They show potential for a variety of biomedical and clinical applications for tissue regeneration, cell and gene therapy. Proper environmental cues are necessary to differentiate MSCs into various tissue types. Laser-induced optical stimulation, low-intensity pulsed ultrasound, mechanical signals, fluid shear stresses, and nanomaterials have been demonstrated to influence their differentiation toward osteoblasts. The ultimate goal of all these strategies is to restore and heal bone loss.

Conventionally, non-union fractures are usually treated with aggressive physical therapy and physical intervention. Studies have indicated that ˜50% of these patients do not have the ability to walk unassisted within 1 year Surgical intervention is expensive, extends recovery time, and doesn't treat the issue. Approximately 6 million fractures each year in the U.S. Of these ˜300,000 are non-union fractures. Most clinical treatments are invasive requiring long recovery. Other conditions involving bone loss can be due to disease or trauma, e.g., diseases such as osteoporosis, osteopenia, and bone cancer (osteosarcoma), and trauma such as defects created by gun shot wounds and mechanical impact suffered during a hit or fall.

FDA-approved anti-resorptive (e.g. bisphosphonates-based), anabolic (parathyroid hormone-based) pharmacological intervention are the current gold standards for the prevention of bone loss due to osteoporosis, but come with potentially severe side-effects. Further, they are systemic interventions not suitable for targeted bone regeneration. Osteoinductive growth factors have been used for certain experimental focal bone regeneration therapies. However, they often are not effective due to rapid diffusion and excretion from the defect site, inefficient delivery (unstable biological activity, short half-life, and minimal tissue penetration). Moreover, the orthopedic implants currently applied for treatment are not designed to work synergistically with these osteoinductive components in order to realize their full biologic potentials. Finally, in humans, high doses of growth factors are required, leading to high costs and limited supply.

Non-pharmacologic strategies, e.g., those based on bone's sensitivity to mechanical/acoustic signals, have been developed for safer bone regeneration. Stimulation techniques utilize mechanical signals generated by sources outside the body such as vibrating plates or piezoelectric ultrasound (US) sources were known in the art. Whole body vibration technologies known in the art rely on weight-bearing ability bones (require standing on a vibrating plate). Additionally, they are unsuitable for targeting specific segments of the skeleton.

The phenomenon where absorption of electromagnetic energy generates acoustic waves is known as the photoacoustic (PA) effect. First demonstrated in 1881 by A. G. Bell, this effect is used for a variety of applications in material science and medicine such as imaging and spectroscopy. An optical (visible and near-infrared lasers) or radio frequency/microwave source is typically used as the electromagnetic source. This source deposits nonionizing electromagnetic energy onto an absorbing surface giving rise to a thermoelastic expansion leading to a wideband ultrasonic emission. This effect forms the basis for emerging bioimaging technologies such as PA microscopy and imaging. Recently, a number of nanomaterials such as single-walled carbon nanotubes (SWNTs) and gold nanoparticles (GNPs) with strong intrinsic absorption at visible/near-infrared (NIR) wavelengths have been used as contrast agents for laser-induced PA imaging.

PA stimulation of MSCs' differentiation toward osteoblasts was demonstrated in cell assays using laser-induced photoacoustic stimulation, which shows that a brief (10 min) daily nanosecond pulse laser-induced PA stimulation enhanced by nanoparticles (SWNTs and GNPs), over 16 days, facilitates MSCs' differentiation toward osteoblasts. See, e.g., Green D E, Longtin J P, Sitharaman B., The effect of nanoparticle-enhanced photoacoustic stimulation on multipotent marrow stromal cells. ACS Nano. 2009; 3(8):2065-72; Balaji Sitharaman, Pramod Avti, Yahfi Talukdar, Kenneth Schaefer and Jon P. Longtin, A Nanoparticle-Enhanced Photoacoustic Stimulus for Bone Tissue Engineering, Tissue Engineering Part A, 2011, 17, 1851-1858.

There remains a need for methods and devices for stimulation of stem or progenitor cells in the body of a patient in a non-invasive manner for treatment or improvement of a condition in the patient.

### SUMMARY OF THE INVENTION

The present invention provides a device for non-invasive stimulation of stem cells and/or progenitor cells in a patient, comprising an acoustic wave generation unit capable of emitting an acoustic wave upon receiving an excitation and an excitation unit comprising an excitation source capable of providing said excitation. The acoustic wave generation unit and the excitation unit are arranged such that the acoustic wave generation unit receives the excitation from the excitation unit and emits acoustic wave to a bodily region comprising stem cells and/or progenitor cells. The frequency of the acoustic wave is preferably in the range of 0.2 to 15 MHz.

The acoustic wave generation unit can comprise a substance, which receives the excitation from the excitation unit. The acoustic wave generation unit can comprise a sealed enclosure, and the substance is contained in the sealed enclosure.

The substance can comprise microparticles and/or nanoparticles and/or dye molecules with good electromagnetic radiation absorption characteristics. The microparticles and/or nanoparticles can be carbon nanotubes, graphene-like nanoparticles, graphitic microparticles, graphitic nanoparticles, gold microparticles, and gold nanoparticles. The dye molecules can be methylene blue and indocyanine.

The acoustic wave generation unit can further comprise a medium, in which the substance is dispersed. The medium can be a cream, ointment, gel or film. The gel can be selected from the group consisting of hyaluranic acid, Fibronectin, chitosan, and polyethylene glycol. The substance can be present in a concentration of 0.01-10 weight percent in the medium.

The acoustic wave generation unit can also comprise a substrate, on which the substance is coated.

The excitation can be an electromagnetic radiation. Preferably, the electromagnetic radiation is a non-ionizing radiation selected from the group consisting of radiofrequency (RF), infrared (IR), and visible. The electromagnetic radiation can be generated by an excitation source is selected from the group consisting of a RF generator, a light emitting diode, and a laser.

Preferably, the excitation source and the acoustic generation unit are arranged such that distance between them is between 0.1 mm-10 cm. Preferably, the acoustic generation unit can form a contact to skin with no air gaps.

The device of the present invention can further comprise an electronic control unit, which is encoded with one or more programs for controlling the acoustic wave generation unit and/or the excitation unit, an user interface, which allows a user to set one or more operational parameters, such as intensity of said acoustic wave, repetition rate, and duration of treatment.

The acoustic wave generation unit is preferably detachable. In one embodiment, the device of the present invention is a wearable device.

The present invention also provides a method for non-invasive stimulation of stem cells and/or progenitor cells in a patient, comprising delivering acoustic waves of a suitable frequency and intensity to a bodily region of the patient comprising the stem cells and/or progenitor cells. The method of the present invention can be carried out by a method comprising placing the device of the present invention to a skin region such that acoustic waves generated by the device can reach the stem cells and/or progenitor cells in the bodily region and providing excitation to acoustic waver generation unit of the device.

The present invention also provides a method for non-invasive bone regeneration in a patient, comprising stimulating osteocyte progenitor cells and/or native bone tissue (resident osteoblasts, vasculature, and extracellular matrix) in a bone region in the patient using the non-invasive acoustic stimulation method and device of the invention. The method can be used for non-invasive treatment of bone loss in a patient by stimulating osteocyte progenitor cells in a bone region suffering from bone loss.

The present invention also provides a method for non-invasive cartilage regeneration in a patient, comprising stimulating chondrocyte progenitor cells in said patient using the non-invasive acoustic stimulation method and device of the invention.

The present invention also provides a method for non-invasive muscle regeneration in a patient, comprising stimulating muscle progenitor cells in the patient using the non-invasive acoustic stimulation method and device of the invention.

The present invention also provides a method for non-invasive nerve regeneration in a patient, comprising stimulating nerve progenitor cells in the patient using the non-invasive acoustic stimulation method and device of the invention.

The present invention further provides a device for non-invasive stimulation of stem cells and/or progenitor cells in a patient, comprising: an acoustic emission means for emitting an acoustic wave upon receiving an excitation; and an excitation means for providing said excitation. In the device, the acoustic emission means and the excitation means are arranged such that the acoustic emission means receives the excitation from the excitation means and emits the acoustic wave to a bodily region of the patient comprising the stem cells and/or progenitor cells. The device can further comprise a controlling means for controlling the acoustic emission means and the excitation means. The controlling means can comprise an electronic circuit encoding one or more programs for controlling the acoustic emission means and/or the excitation means. The device can further comprise a displaying means for displaying one or more operational parameters of the device and/or an inputting means for inputting one or more commands to set the one or more operational parameters. The device can further comprise means for securing said device to a patient.

### BRIEF DESCRIPTION OF THE FIGURES

FIG. 1 is an illustration of an embodiment of non-invasive stimulation of stem or progenitor cells in the body of a patient. Electromagnetic radiation stimulates nanoparticles to produce acoustic waves, which is transmitted through the skin and tissue to induce fate of pluripotent cells into osteoblasts. Surface adhesion molecules (integrins) are activated and promote migration of cells to the wound area.

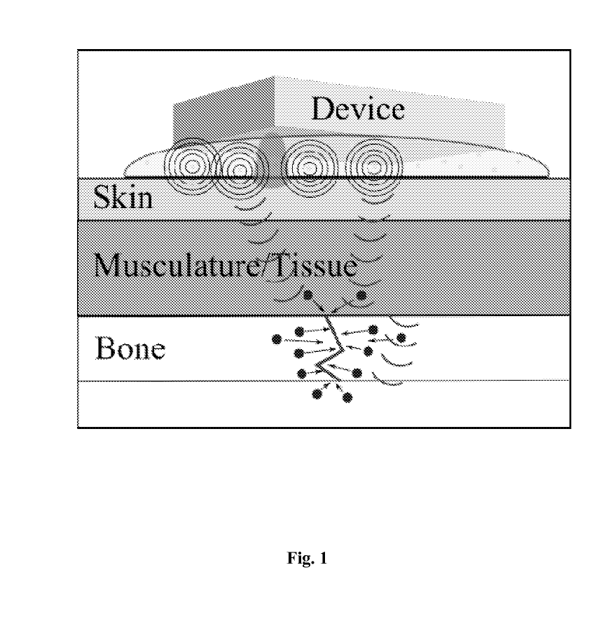

FIG. 2 shows a wearable device including consumables of the present invention.

### DETAILED DESCRIPTION OF THE INVENTION

The present invention provides a method for non-invasive acoustic stimulation in a bodily region in a patient (e.g., a injured region) stem cells, progenitor cells, native osteoblasts, extracellular matrix signals responsible for healing, and/or blood vessels that would increase blood flow into the bodily region by delivering acoustic waves of a suitable frequency and intensity to the bodily region of the patient. The present invention also provides a device for non-invasive stimulation of stem cells, progenitor cells, native osteoblasts, extracellular matrix signals responsible for healing, and/or blood vessels in the patient by generating and delivering acoustic waves of a suitable frequency and intensity to the stem cells, progenitor cells, native osteoblasts, extracellular matrix signals responsible for healing, and/or blood vessels.

The patient can be any animal, including but not limited to a mammal. In a preferred embodiment, the patient is a human.

Acoustic stimulation using the method and device of the present invention do not require external (e.g., weight bearing) or internal (e.g. muscle) forces, and can be applied in any direction on any tissue location of the body and any bone regions of the skeleton. It can be focused and applied to specific regions or segments of the tissue or bone that are in need of additional growth, e.g., additional bone mass and/or strength (e.g., bone tissues that are not predominantly weight bearing, such as the distal forearm or lower extremities of bedridden patients). Further, acoustic stimulation allows a biophysical rather than biochemical strategy for osteoinduction. Additionally, the same conditions allow imaging and therapy, whereas in other method such as conventional ultrasonic method, the same conditions do not allow simultaneous imaging and therapy, e.g., high intensity (in MHz frequency) is required for imaging and low intensity (in KHz frequency) is required for therapy.

As used herein, the term “non-invasive” refers to a procedure that does not involve breaking the skin of the patient. For example, in the present invention, the acoustic waves can be applied to a skin region in close proximity to the bodily region containing the stem cells and/or progenitor cells and transmitted to the stem cells and/or progenitor cells through the skin and tissues.

As used herein, the term “stimulation” is contemplated to refer to any enhancement of the differentiation and/or growth or proliferation of stem cells or progenitor cells as compared to a level of differentiation and/or growth or proliferation without using the method of the present invention. Thus, stimulation includes, but is not limited to, induction and improvement of the differentiation and/or growth of the cells.

The method of the present invention can be applied to stem cells or progenitor cells in any bodily region of the patient so long as acoustic waves can reach the region with sufficient intensity. For example, the method of the present invention can be applied to bones, connective tissues, muscles, and nervous tissues. The stem cells or progenitor cells can be cells native to the particular bodily region, or implanted or injected cells. For example, the stem cells or progenitor cells can be implanted as part of a tissue or prosthesis. In cases that the stem cells or progenitor cells are introduced into the patient prior to the application of the method of the present invention, e.g., by injection or implant, acoustic stimulation of the stem cells or progenitor cells can be performed using the method of the present invention non-invasively after the injection or implant.

The stem cells or progenitor cells can be any type of stem cells or progenitor cells. Of particular interest are mesenchymal stem cells (MSCs) which can differentiate, in vitro or in vivo, into a variety of connective tissue cells or progenitor cells, including, but not limited to, including mesodermal (osteoblasts, chondrocytes, tenocytes, myocytes, and adipocytes), ectodermal (neurons, astrocytes) and endodermal (hepatocytes) derived lineages. The terms “mesenchymal stem cell” and “marrow stromal cell” are often used interchangeably, so it is important to note that MSCs encompass multipotent cells from sources other than marrow, including, but not limited to, muscle, dental pulp, cartilage, synovium, synovial fluid, tendons, hepatic tissue, adipose tissue, umbilical cord, and blood, including cord blood. Also of interest are embryonic stem (ES) cells, which can be differentiated into all cell types.

In the present invention, non-invasive stimulation of stem cells and/or progenitor cells in a patient can be carried out by using a device that generates and emits acoustic waves. In one embodiment, the device comprises an acoustic wave generation unit capable of emitting acoustic waves of a suitable frequency and intensity upon receiving an excitation. The device can be placed at an appropriate skin region such that acoustic waves generated by the acoustic wave generation unit can reach the target bodily region containing the stem cells and/or progenitor cells. The device may also contain an excitation unit which serves as an excitation source to the acoustic wave generation unit.

In the device of the present invention, the acoustic wave generation unit can comprise a substance that receives the excitation from the excitation unit. The acoustic wave generation unit can also comprise a medium in which the substance is dispersed. The medium can be, but is not limited to, a cream, an ointment, a gel and a film. The amount of the substance in the medium depends on various properties of the substance and the medium, such as the absorption coefficient of the substance and elastic constant of the medium, as well as the excitation used and the desired frequency and intensity of the generated acoustic waves. A person skilled in the art would be able to select the appropriate amount based on routine experimentation. In one embodiment, the acoustic wave generation unit comprises the substance in a concentration of 0.01-10 weight percent (weight percent of substance=(weight of substance in grams)/(total weight of substance and medium in grams)×100.

In one embodiment, the medium is a gel selected from the group consisting of hyaluranic acid, Fibronectin, chitosan, and polyethylene glycol.

The substance and/or the medium can be placed in a sealed enclosure, e.g., an enclosure that allows transmission of the excitation into the enclosure and transmission of the acoustic waves from the enclosure but not transmission of the substance in and out of the enclosure.

The acoustic wave generation unit can also comprise a substrate on at least one surface of which the substance is coated. The substrate can be made of any suitable material known in the art. The substrate is preferably a thin sheet made from a transparent material such as glass or a polymeric material. Suitable polymeric material include, but is not limited to, Polyethylene terephthalate (PET), Polyethylene Naphthalate (PEN) and polycarbonate polymers. In one embodiment, the substance such as the nanoparticles and/or dye molecules can be coated on the substrate with a thickness between 10-1000 nm. In one embodiment, the substrate is coated on one side of the surface with the other side for contact with the patient skin. In another embodiment, the substrate is coated on the side that contacts with the patient skin.

Thus, suitable acoustic wave generation units include, but are not limited to, a pad, a patch, a film, and sheet that can be attached to the skin. Preferably, the acoustic wave generation unit is attached to the skin in a manner to reduce or prevent air bubbles at the interface. The acoustic wave generation unit of the present invention can be a disposable unit, which can be detached from the excitation unit.

In one embodiment, a skin care substance layer is coated on the acoustic wave generation unit on the side that contacts the skin. In the embodiment in which the substance such as the nanoparticles or dye molecules is coated on the side the substrate that contacts the skin, the skin care substance layer can be coated on top of the coated layer of the substance.

In one embodiment, the acoustic wave generation unit is in the form of a thin film cover which can be attached to the excitation unit. The thin film contains the substance such as nanoparticles and/or dye molecules between 10-1000 nm thickness coated on a transparent glass substrate or a polymer substrate such as Polyethylene terephthalate (PET), Polyethylene Naphthalate (PEN) and polycarbonate polymers. When used for treatment, the substrate cover is attached to the excitation unit, and the device is brought into a suitable contact with the skin by the substrate.

In another embodiment, the film is a plastic or elastomer sheet that is coated with the substance, e.g., SWNTs or nanoparticles. The film can be a disposable transparent sheet coated on one side (not touching the skin) with the substance, e.g., nanoparticles or SWNTs. The wearable device is attached to the patient via the film. The device is then turned on to emit light or laser pulses to the film, and the acoustic waves generated by the nanoparticle or SWNTs coated film are transmitted into the patient to the site of treatment, e.g., the site of injury.

In still another embodiment, the acoustic wave generation unit is in the form of a gel pad or gel cover which can be attached to the excitation unit. The gel pad or gel cover contains the substance such as nanoparticles and/or dye molecules dispersed in a gel. When used for treatment, the gel pad or gel cover is attached to the excitation unit, and the device is brought into a suitable contact with the skin by the gel pad or gel cover.

The acoustic wave generation unit and the excitation unit can be configured in any manner as long as the acoustic wave generation unit receives the excitation from the excitation unit and emits the acoustic waves. A person skilled in the art will readily be able to configure the acoustic wave generation unit and the excitation unit based on the characteristics of both units. In one embodiment, the excitation unit and the acoustic wave generation unit are arranged such that distance between them is between 0.1 mm-10 cm to facilitate absorption of the electromagnetic energy.

The device of the present invention can also have straps or other forms of securing meaning for securing the device on the patient's body. Thus, the device of the present invention can be a wearable device.

In preferred embodiments of the invention, the excitation is an electromagnetic radiation. Preferably, the electromagnetic radiation is a non-ionizing radiation selected from the group consisting of radiofrequency (RF), infrared (IR), and visible. Suitable excitation sources for generating the electromagnetic radiation include, but are not limited to, a RF generator, a light emitting diode, and a laser. The substance absorbs the electromagnetic radiation and generates acoustic waves.

Preferably, the substance exhibits strong electromagnetic absorption properties at the electromagnetic wavelength provided by the excitation unit. Suitable substances that can be used in the present invention include, but are not limited to, microparticles, nanoparticles, and dye molecules.

In a preferred embodiment of the present invention, the substance comprises microparticles and/or nanoparticles which absorb the electromagnetic radiation and generates acoustic waves. The microparticles and/or nanoparticles can be of various size and composition, so long as they can absorb electromagnetic energy from the excitation unit to generate acoustic (mechanical) energy. The electromagnetic absorbance properties of the microparticles and/or nanoparticles result from the composition of the microparticles and/or nanoparticles themselves or from moieties linked to the microparticles and/or nanoparticles. In one embodiment, the nanoparticles can be composed of a variety of substances, including metals such as gold, platinum, silver, and titanium. The microparticles and nanoparticles can further include carbon nanoparticles, including but not limited to carbon nanotubes, single walled carbon nanotubes (SWNTs), graphene-like nanoparticles, and graphitic microparticles and/or nanoparticles. As used herein, the term “graphene-like nanoparticle” refers to a carbon nanoparticle comprising one or more atomic carbon sheets or layers. A graphene-like nanoparticle can be a carbon nanoplatelet or a carbon nanoribbon. Nanoparticles of the invention also include nanotubes composed of, for example, boron nitride. Also, as mentioned, desired absorbance properties can be obtained by linking sensitizing dyes to the microparticles and/or nanoparticles. In one embodiment exemplified herein, the nanoparticles are gold nanoparticles. In another embodiment exemplified herein, the nanoparticles are single walled carbon nanotubes. The nanoparticles of the invention can be relatively homogenous in size and shape, or be variable.

In another preferred embodiment of the present invention, the substance comprises dye molecules selected from the group consisting of methylene blue and indocyanine which absorb the electromagnetic radiation and generates acoustic waves.

According to the invention, electromagnetic radiation over a wide range of frequencies can be used to induce acoustic waves. In one embodiment of the invention, high frequency (HF) electromagnetic radiation (about 3 MHz to about 30 MHz) is selected. In another embodiment of the invention, very high frequency (VHF) electromagnetic radiation (about 30 MHz to about 300 MHz) is selected. In another embodiment of the invention, ultra high frequency (UHF) electromagnetic radiation (about 300 MHz to about 3 GHz) is selected. In another embodiment of the invention, super high frequency (SHF) electromagnetic radiation (about 3 GHz to about 30 GHz) is selected. In another embodiment of the invention, extremely high frequency (EHF) electromagnetic radiation (about 30 GHz (1 cm) to about 300 GHz (1 mm)) is selected. In other embodiments, infrared radiation is selected such as, for example, far infrared (about 300 GHz (1 mm) to about 30 THz (10 μm)), mid-infrared (about 30 THz (10 μm) to about 120 THz (2.5 μm)), or near infrared (about 120 THz (2.5 μM) to about 400 THz (750 nm)). In other embodiments, electromagnetic radiation in the visible region (about 400 nm to about 700 nm) or in the ultraviolet region (about 50 nm to about 400 nm) is selected. In certain embodiments, the electromagnetic radiation is coherent (e.g., generated by a laser). In this regard, methods of the invention can often be facilitated by using electromagnetic fields generated by equipment already in use in hospitals and health care facilities. For example, the RF range around 40-50 MHz is used in nuclear magnetic resonance (NMR) and typical magnetic resonance imaging (MRI) uses frequencies from under 1 MHz up to about 400 MHz. Some examples include 13.56 MHz, 42.58 MHz (1-T scanner) and 63.86 MHz (1.5-T scanner). In one example disclosed herein, SWNTs were irradiated with SHF electromagnetic radiation (about 3 GHz). Infrared, visible, and ultraviolet light sources can also be used for stimulation. Commonly used wavelengths include, but are not limited to, 532 nm, 633 nm, 764 nm, and 1064 nm. In another example, gold nanoparticles were illuminated with coherent visible light (532 nm).

According to the invention, the radiation can be pulsed in a manner that results in pulsed acoustic waves and avoids heating of the acoustic wave generation unit. For example, the electromagnetic radiation can be pulsed at a frequency from about 5 to about 500 Hz, or from about 10 Hz to about 100 Hz. In one example, 3 GHz radiation was pulsed at 100 pulses/sec. with a pulse duration of 0.5 μs. In another example, a 532 nm laser was pulsed at a rate of 10 pulses/sec. with a pulse duration of 200 ns. Heating can also be reduced or prevented by limiting the intensity of the electromagnetic radiation.

Preferably, the frequency of the acoustic waves is in the range of 0.2 to 15 MHz.

In a specific embodiment, the source of the electromagnetic radiation is a light emitting diode (LED) having a radiation wavelength in the range of 350-950 nm. In one embodiment, the radiation is pulsed radiation having a pulse duration of 10-100 ns. In another embodiment, the radiation has a pulse repetition rate of 10-50,000 Hz. In still another embodiment, the LED has a peak power of 1-75 W.

Preferably, the device of the present invention also comprises an electronic control unit for controlling the acoustic wave generation unit and/or the excitation unit. The electronic control unit can contain any electronic controlling means, e.g., including one or more hardware and/or software modules for executing one or more programs for controlling the operation of the acoustic wave generation unit and/or the excitation unit. For example, the electronic control unit can comprise a microcontroller or microprocessor and an appropriate amount of memory for execution of the one or more programs. The electronic control unit can also comprise a storage unit such as ROM or a disk drive for storing the one or more programs and/or data such as device parameters. The electronic control unit can also comprise one or more removable media drives for importing and exporting data and programs from one or more removable media, including but not limited to, optical discs, e.g., Blu-ray discs, DVDs, CDs, memory cards, e.g., compact flash card, secure digital card, and USB drives. The electronic control unit can also comprise a network connecting means such as a network card for connecting to a local network and/or the internet. The electronic control unit can further comprise a wireless remote control means to allow remote control of the operation of the device. The wireless remote control means can include a receiver, which can be included in the electronic control unit, and a transmitter, which can be a stand alone remote.

The device of the present invention can further comprise a user interface, which allows a user to set one or more operational parameters. The one or more operational parameters include, but are not limited to, intensity of the acoustic wave, the repetition rate of the acoustic pulse, and duration of treatment. A person skilled in the art would be able to select the appropriate operational parameters based on, e.g., the stem or progenitor cells involved, the location of the cells, and the nature of the treatment.

The user interface can include any suitable displaying means known in the art, such as a LCD, a set of LED, for displaying the device status information and/or the user inputs. The user interface can also include a suitable inputting means, such as one or more keys or a key pad, for inputting one or more operational parameters.

In one embodiment, the method and device of the present invention is used for bone regeneration by non-invasive acoustic stimulation of osteocyte progenitor cells. Thus, the present invention provides a method and device for treating bone loss or bone fracture in a patient. Bone loss and bone fracture can be due to disease or trauma, e.g., diseases such as osteoporosis, osteopenia, and bone cancer (osteosarcoma), and trauma such as defects created by gun shot wounds and mechanical impact suffered during a hit or fall. The present invention can be used for treating bone loss and/or improving bone healing for patients suffering from any of these conditions. The acoustic wave generation unit is placed in contact with a skin region in proximity to the bone or bone region that suffers from bone loss or bone fracture. When the excitation unit is activated, acoustic waves are generated and transmitted through skin and tissue to the osteocyte progenitor cells in the bone or bone region. The treatment can be performed for a desired treatment duration, and repeated for additional treatments. A suitable treatment schedule can be determined by a person skilled in the art, e.g., a medical practitioner, based on the condition of the patient and the goal of the treatment.

In another embodiment, the method and device of the invention are used on specific segments of the human skeleton as an anabolic or anti-catabolic non-pharmacological prophylaxis and/or therapeutic intervention to improve bone quantity and quality. As a prophylaxis, it reduces healthcare costs by reducing the incidences of fractures related to bone loss in the elderly, and post menopausal women. As a therapeutic intervention, it accelerates bone regeneration reducing treatment time and costs. Thus, in one embodiment, the method and device of the invention are used for osteo-integration, accelerate fracture healing, and treatment of segmental bone defects with bone tissue engineering strategies.

In another embodiment, the method and device of the present invention is used for cartilage regeneration in a patient by non-invasive acoustic stimulation of chondrocyte progenitor cells. The acoustic wave generation unit is placed in contact with a skin region in proximity to the bodily region where cartilage regeneration is desired. When the excitation unit is activated, acoustic waves are generated and transmitted through skin and tissue to the chondrocyte progenitor cells in the bodily region. The treatment can be performed for a desired treatment duration, and repeated for additional treatments. A suitable treatment schedule can be determined by a person skilled in the art, e.g., a medical practitioner, based on the condition of the patient and the goal of the treatment.

In still another embodiment, the method and device of the present invention is used for muscle regeneration in a patient by non-invasive acoustic stimulation of muscle progenitor cells. The acoustic wave generation unit is placed in contact with a skin region in proximity to the muscle region where regeneration is desired. When the excitation unit is activated, acoustic waves are generated and transmitted through skin and tissue to the muscle progenitor cells in the muscle region. The treatment can be performed for a desired treatment duration, and repeated for additional treatments. A suitable treatment schedule can be determined by a person skilled in the art, e.g., a medical practitioner, based on the condition of the patient and the goal of the treatment.

In still another embodiment, the method and device of the present invention is used for nerve regeneration in a patient by non-invasive acoustic stimulation of nerve progenitor cells. The acoustic wave generation unit is placed in contact with a skin region in proximity to the nerve region where regeneration is desired. When the excitation unit is activated, acoustic waves are generated and transmitted through skin and tissue to the nerve progenitor cells in the nerve region. The treatment can be performed for a desired treatment duration, and repeated for additional treatments. A suitable treatment schedule can be determined by a person skilled in the art, e.g., a medical practitioner, based on the condition of the patient and the goal of the treatment.

In still another embodiment, the method and device of the present invention is used in conjunction with an implant. The implant can be a metal implant, such as an artificial hip, knee, or shoulder, to which bone must meld. Other examples include dental implants. The implant can also be made of a composite material such as a fiber composite. For example, along with carbon fiber and fiberglass composites, orthopedic implants can be made from composite materials. The implants can be implanted directly, or incubated with osteoblasts from the recipient prior to implantation. The implants and their neighboring regions are then subjected to acoustic stimulation according to the present invention.

When implanted or injected, stem cell development is often governed by the site of implantation or the site in the body to which the stem cells home. According to the invention, differentiation of stem cells and progenitor cells can also be directed in vitro prior to implantation by selection of media components and/or matrix components. For example, cytokines and growth factors that promote osteogenic differentiation include various isoforms of bone morphogenetic protein (BMP) such as BMP-2, -6, and -9, interleukin-6 (IL-6), growth hormone, and others. (See, e.g., Heng et al., 2004, J. Bone Min. Res. 19, 1379-94). Cytokines and growth factors that promote chondrogenesis include various isoforms of TGF-β and bone morphogenetic protein, activin, FGF, and other members of the TGF-β superfamily. Chemical factors that promote osteogenesis and chondrogenesis to include prostaglandin E2, dexamethasone. Osteogenesis or chondrogenesis can also be favored by selection of extracellular matrix (ECM) material. For example, chondrogenesis is favored by naturally occurring or synthetic cartilage extracellular matrix (ECM). Such an ECM can comprise collagenous proteins such as collagen type II, proteoglycans such as aggrecan, other proteins, and hyaluronan. (See, e.g., Heng et al., 2004, Stem Cells 22, 1152-67). Phenotypic markers expressed by cells of the various lineages are well known in the art.

The method and device of the present invention can also be employed to non-invasively inhibit differentiation of adipocyte progenitor cells to adipocytes in a patient. As used herein, inhibition of differentiation to adipocytes means that differentiation is reduced, but not necessarily prevented entirely. In an embodiment of the invention, the acoustic wave generation unit can be applied on the skin, for example in a cream or ointment, or embedded in a film, patch or other covering that is applied near the fat tissue. Electromagnetic excitation is then applied to induce acoustic stimulation of the tissue.

### EXAMPLES

The following examples are presented by way of illustration of the present invention, and are not intended to limit the present invention in any way.

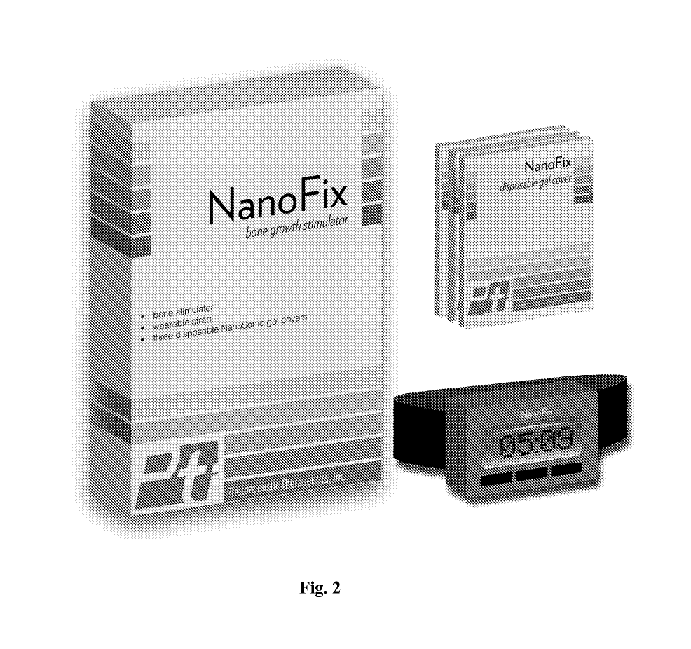

A low cost device is designed and developed with the following specifications with a prototype cost <$500 (FIG. 2).

Technical Specifications of the electronic part of the device including the excitation unit and electronic control unit:

Hardware:

| | |
|---|---|
| Microcontroller | 32 bit high speed microcontroller |
| ROM | 64 KB |
| RAM | 8 KB |
| Display | 8 characters × 1 line LCD monochrome with blacklight |
| | or some equivalent better option. |
| Keypad | 4 keys |
| Light source | HORIBA nanoLED light source or OSRAM LASER |
| | light source or equivalent |
| Power supply | 9 V rechargeable battery operated with |
| | 110 VAC/230 VAC power adaptor for recharging |

Firmware:

| | |
|---|---|
| Repetition rate | Selectable repetition rate from 10 Hz to 100 Hz |
| Pulse width | Fixed pulse width depending on light source module |
| | selected |
| Auto shut off | Selectable time in minutes for auto shut off facility |
| Treatment time | Selectable in minutes |
| Battery status | Indication for battery status |

Features:

1. Low power battery operated
2. Device will generate trigger pulse with set repetition rate and fixed
pulse width to drive HORIBA/OSRAM/Equivalent light source. The light
source emits in the near infrared region (500 nm to 1400 nm).
3. Instrument will be portable & strap-able.

A nanoparticle gel formulation is prepared by sol-gel processing, wherein the nanoparticle, pectin and mono or disaccharides are used. First, an aqueous solution of the nanoparticle is prepared at concentrations between 0.1-10 mg/ml. Next, a separate aqueous solution comprising mono or disaccharides is prepared at concentrations between 0.1-10 mg/ml. The two solutions are mixed at different ratios to form a third solution at a temperature from about 80 to 100° C. Finally, the combined solution is incubated at an elevated temperature of about 50 to 180° C. for 1-5 hours in order to cause gelation. This nanoparticle gel formulation is filled in non-permeable transparent plastic packets to form the gel pads.

A thin film of a non-permeable transparent substrate (sheet) made of glass or a polymer substrate such as Polyethylene terephthalate (PET), Polyethylene Naphthalate (PEN) or polycarbonate polymer is coated on one side with nanoparticles or dyes by vacuum filtration, spray coating, bar coating, or spin coating. The nanometer thin (film) layer 10-1000 nm is composed of gold particles, carbon naparticles or dyes. A skin care substance layer is coated on the at least one nanometer metal layer.

All references cited herein are incorporated by reference in their entireties and for all purposes to the same extent as if each individual publication or patent or patent application was specifically and individually indicated to be incorporated by reference in its entirety for all purposes.

As will be apparent to those skilled in the art, many modifications and variations of the present invention can be made without departing from its spirit and scope. The specific embodiments described herein are offered by way of example only, and the invention is to be limited only by the terms of the appended claims along with the full scope of equivalents to which such claims are entitled.

1. A device for non-invasive stimulation of stem cells and/or progenitor cells in a patient, comprising:
a) an acoustic wave generation unit capable of emitting an acoustic wave upon receiving an excitation; and
b) an excitation unit comprising an excitation source capable of providing said excitation,
wherein said acoustic wave generation unit and said excitation unit are arranged such that said acoustic wave generation unit receives said excitation from said excitation unit and emits said acoustic wave to a bodily region comprising said stem cells and/or progenitor cells.

2. The device for non-invasive stimulation of stem cells and/or progenitor cells in a patient of claim 1, wherein said acoustic wave generation unit comprises a substance, said substance receives said excitation from said excitation unit.

3. The device for non-invasive stimulation of stein cells and/or progenitor cells in a patient of claim 2, wherein said acoustic wave generation unit comprises a sealed enclosure, and wherein said substance is contained in said sealed enclosure.

4. The device for non-invasive stimulation of stem cells and/or progenitor cells in a patient of claim 2, wherein said excitation is an electromagnetic radiation, and wherein said substance absorbs said electromagnetic radiation.

5. The device for non-invasive stimulation of stem cells and/or progenitor cells in a patient of claim 4, wherein said substance comprises microparticles and/or nanoparticles and/or dye molecules.

6. The device for non-invasive stimulation of stem cells and/or progenitor cells in a patient of claim 5, wherein said microparticles and/or nanoparticles are selected from the group consisting of carbon nanotubes, graphene-like nanoparticles, graphitic microparticles, graphitic nanoparticles, gold microparticles, and gold nanoparticles.

7. The device for non-invasive stimulation of stein cells and/or progenitor cells in a patient of claim 5, wherein said dye molecules are selected from the group consisting of methylene blue and indocyanine.

8. The device for non-invasive stimulation of stem cells and/or progenitor cells in a patient of claim 4, wherein said acoustic wave generation unit further comprises a medium, and wherein said substance is dispersed in said medium.

9. The device for non-invasive stimulation of stem cells and/or progenitor cells in a patient of claim 8, wherein said medium is a cream, ointment, gel or film.

10. The device for non-invasive stimulation of stein cells and/or progenitor cells in a patient of claim 9, wherein said gel is selected from the group consisting of hyaluranic acid, Fibronectin, chitosan, and polyethylene glycol.

11. The device for non-invasive stimulation of stem cells and/or progenitor cells in a patient of claim 8, wherein said acoustic wave generation unit comprises said substance in a concentration of 0.01-10 weight percent in said medium.

12. The device for non-invasive stimulation of stem cells and/or progenitor cells in a patient of claim 4, wherein said acoustic wave generation unit comprises a substrate, and wherein said substance is coated on said substrate.

13. The device for non-invasive stimulation of stem cells and/or progenitor cells in a patient of claim 4, wherein said electromagnetic radiation is a non-ionizing radiation selected from the group consisting of radiofrequency (RF), infrared (IR), and visible.

14. The device for non-invasive stimulation of stem cells and/or progenitor cells in a patient of claim 13, wherein said excitation unit comprises an excitation source selected from the group consisting of a RF generator, a light emitting diode, and a laser.

15. The device for non-invasive stimulation of stem cells and/or progenitor cells in a patient of claim 12, wherein said excitation source and said acoustic generation unit are arranged such that distance between them is between 0.1 mm-10 cm, and said acoustic generation unit can form a contact to skin with no air gaps.

16. The device for non-invasive stimulation of stem cells and/or progenitor cells in a patient of claim 1, further comprising
c) an electronic control unit, wherein said electronic control unit is encoded with one or more programs for controlling said acoustic wave generation unit and/or said excitation unit.

17. The device for non-invasive stimulation of stein cells and/or progenitor cells in a patient of claim 1, further comprising
d) an user interface, wherein said user interface allows a user to set one or more operational parameters.

18. The device for non-invasive stimulation of stein cells and/or progenitor cells in a patient of claim 17, wherein said one or more operational parameters are selected from the group consisting of intensity of said acoustic wave, repetition rate, and duration of treatment.

19. The device for non-invasive stimulation of stein cells and/or progenitor cells in a patient of claim 1, wherein said frequency of said acoustic wave is in the range of 0.2 to 15 MHz.

20. The device for non-invasive stimulation of stem cells and/or progenitor cells in a patient of claim 1, wherein said acoustic wave generation unit is detachable.

21. The device for non-invasive stimulation of stem cells and/or progenitor cells in a patient of claim 1, which is a wearable device.

22. A method for non-invasive stimulation of stein cells and/or progenitor cells in a patient, comprising delivering acoustic waves of a suitable frequency and intensity to a bodily region of said patient comprising said stem cells and/or progenitor cells.

23. The method for non-invasive stimulation of stem cells and/or progenitor cells in a patient of claim 22, wherein said delivering is carried out by a method comprising
i) placing a device comprising an acoustic wave generation unit to a skin region such that acoustic waves generated by said acoustic wave generation unit can reach said stem cells and/or progenitor cells in said bodily region, wherein said acoustic wave generation unit is capable of emitting acoustic waves of said frequency and intensity upon receiving an excitation; and
ii) providing said excitation to said acoustic waver generation unit.

24. The method for non-invasive stimulation of stem cells and/or progenitor cells in a patient of claim 23, wherein said providing said excitation is carried out by an excitation unit comprising an excitation source capable of providing said excitation, wherein said acoustic wave generation unit and said excitation unit are arranged such that said acoustic wave generation unit receives said excitation from said excitation unit and emits said acoustic wave.

25. The method for non-invasive stimulation of stein cells and/or progenitor cells in a patient of claim 23, wherein said acoustic wave generation unit comprises a substance, said substance receives said excitation from said excitation unit.

26. The method for non-invasive stimulation of stem cells and/or progenitor cells in a patient of claim 25, wherein said acoustic wave generation unit comprises a sealed enclosure, and wherein said substance is contained in said sealed enclosure.

27. The method for non-invasive stimulation of stem cells and/or progenitor cells in a patient of claim 26, wherein said excitation is an electromagnetic radiation, and wherein said substance absorbs said electromagnetic radiation.

28. The method for non-invasive stimulation of stem cells and/or progenitor cells in a patient of claim 27, wherein said substance comprises microparticles and/or nanoparticles and/or dye molecules.

29. The method for non-invasive stimulation of stem cells and/or progenitor cells in a patient of claim 28, wherein said microparticles and/or nanoparticles are selected from the group consisting of carbon nanotubes, graphene-like nanoparticles, graphitic microparticles, graphitic nanoparticles, gold microparticles, and gold nanoparticles.

30. The method for non-invasive stimulation of stem cells and/or progenitor cells in a patient of claim 28, wherein said dye molecules are selected from the group consisting of methylene blue and indocyanine.

31. The method for non-invasive stimulation of stem cells and/or progenitor cells in a patient of claim 27, wherein said acoustic wave generation unit further comprises a medium, and wherein said substance is dispersed in said medium.

32. The method for non-invasive stimulation of stem cells and/or progenitor cells in a patient of claim 31, wherein said medium is a cream, ointment, gel or film.

33. The method for non-invasive stimulation of stem cells and/or progenitor cells in a patient of claim 32, wherein said gel is selected from the group consisting of hyaluranic acid, Fibronectin, chitosan, and polyethylene glycol.

34. The method for non-invasive stimulation of stem cells and/or progenitor cells in a patient of claim 31, wherein said acoustic wave generation unit comprises said substance in a concentration of 0.01-10 weight percent in said medium.

35. The method for non-invasive stimulation of stem cells and/or progenitor cells in a patient of claim 27, wherein said acoustic wave generation unit comprises a substrate, and wherein said substance is coated on said substrate.

36. The method for non-invasive stimulation of stem cells and/or progenitor cells in a patient of claim 27, wherein said electromagnetic radiation is a non-ionizing radiation selected from the group consisting of radiofrequency (RF), infrared (IR), and visible.

37. The method for non-invasive stimulation of stem cells and/or progenitor cells in a patient of claim 36, wherein said excitation unit comprises an excitation source selected from the group consisting of a RF generator, a light emitting diode, and a laser.

38. The method for non-invasive stimulation of stem cells and/or progenitor cells in a patient of claim 35, wherein said excitation source and said acoustic generation unit are arranged such that distance between them is between 0.1 mm-10 cm, and said acoustic generation unit contacts the skin with no air gaps.

39. The method for non-invasive stimulation of stem cells and/or progenitor cells in a patient of claim 22, said device further comprising
c) an electronic control unit, wherein said electronic control unit is encoded with one or more programs for controlling said acoustic wave generation unit and/or said excitation unit.

40. The method for non-invasive stimulation of stem cells and/or progenitor cells in a patient of claim 22, said device further comprising
d) an user interface, wherein said user interface allows a user to set one or more operational parameters.

41. The method for non-invasive stimulation of stem cells and/or progenitor cells in a patient of claim 40, wherein said one or more operational parameters are selected from the group consisting of intensity of said acoustic waves, repetition rate, and duration of treatment.

42. The method for non-invasive stimulation of stem cells and/or progenitor cells in a patient of claim 22, wherein said frequency of said acoustic wave is in the range of 0.2 to 15 MHz.

43. The method for non-invasive stimulation of stem cells and/or progenitor cells in a patient of claim 22, wherein said acoustic wave generation unit is detachable.

44. The method for non-invasive stimulation of stem cells and/or progenitor cells in a patient of claim 22, wherein said device is a wearable device.

45. A method for non-invasive bone regeneration in a patient, comprising stimulating osteocyte progenitor cells in a bone region in said patient by the method for non-invasive stimulation of stem cells and/or progenitor cells in a patient of claim 22.

46. A method for non-invasive treatment of bone loss in a patient, comprising stimulating osteocyte progenitor cells in a bone region suffering from bone loss by the method for non-invasive stimulation of stem cells and/or progenitor cells in a patient of claim 22.

47. A method for non-invasive cartilage regeneration in a patient, comprising stimulating chondrocyte progenitor cells in said patient by the method for non-invasive stimulation of stein cells and/or progenitor cells in a patient of claim 22.

48. A method for non-invasive muscle regeneration in a patient, comprising stimulating muscle progenitor cells in said patient by the method for non-invasive stimulation of stem cells and/or progenitor cells in a patient of claim 22.

49. A method for non-invasive nerve regeneration in a patient, comprising stimulating nerve progenitor cells in said patient by the method for non-invasive stimulation of stem cells and/or progenitor cells in a patient of claim 22.

50. A device for non-invasive stimulation of stem cells and/or progenitor cells in a patient, comprising:
a) an acoustic emission means for emitting an acoustic wave upon receiving an excitation; and
b) an excitation means for providing said excitation,
wherein said acoustic emission means and said excitation means are arranged such that said acoustic emission means receives said excitation from said excitation means and emits said acoustic wave to a bodily region comprising said stem cells and/or progenitor cells.

51. The device for non-invasive stimulation of stem cells and/or progenitor cells in a patient of claim 50, further comprising
c) a controlling means for controlling said acoustic emission means and said excitation means.

52. The device for non-invasive stimulation of stem cells and/or progenitor cells in a patient of claim 51, wherein said controlling means comprises an electronic circuit encoding one or more programs for controlling said acoustic emission means and/or said excitation means.

53. The device for non-invasive stimulation of stein cells and/or progenitor cells in a patient of claim 51, further comprising
d) a displaying means for displaying one or more operational parameters of said device and/or an inputting means for inputting one or more commands to set said one or more operational parameters.

54. The device for non-invasive stimulation of stem cells and/or progenitor cells in a patient of claim 53, further comprising
e) means for securing said device to a patient.
